SEQUENCE LISTING (36 sequences)

SEQ ID NO: 1 - DNA, 23 bp, Artificial
  Part of oligo pair for isolating pgk1 promoter from S. cerevisiae
agattcctgacttcaactcaaga

SEQ ID NO: 2 - DNA, 54 bp, Artificial
  Part of oligo pair for isolating pgk1 promoter from S. cerevisiae
actcgagagatcttgatcagaattcttgttttatatttgttgtaaaaagtagat

SEQ ID NO: 3 - DNA, 49 bp, Artificial
  Part of oligo pair for isolating cyc1 transcription terminator from S. cerevisiae
aagaattctgatcaagatctctcgagtatctagagggccgcatcatgta

SEQ ID NO: 4 - DNA, 22 bp, Artificial
  Part of oligo pair for isolating cyc1 transcription terminator from S. cerevisiae
taccgcctttgagtgagctgat

SEQ ID NO: 5 - DNA, 31 bp, Artificial
  Part of oligo pair for fusing pgk1 promoter and cyc1 transcription terminator
aggatccacatctgcataataggcatttgca

SEQ ID NO: 6 - DNA, 35 bp, Artificial
  Part of oligo pair for fusing pgk1 promoter and cyc1 transcription terminator
ttctagagcatgcttcattaatgcagggccgcaaa

SEQ ID NO: 7 - DNA, 1040 bp, Artificial
  pgk1-restriction endonuclease (EcoRI-BclI-BglII-XboI)-transcription terminator cyc1 fragment
acatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaagaattctgatcaagatctctcgagtatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaa

SEQ ID NO: 8 - DNA, 25 bp, Artificial
  Part of oligo pair for isolating Act1 promoter
agacacacgcgagaacatatataca

SEQ ID NO: 9 - DNA, 50 bp, Artificial
  Part of oligo pair for isolating Act1 promoter
tgagctcggtaccaagcttgttaattcagtaaattttcgatcttgggaag

SEQ ID NO: 10 - DNA, 44 bp, Artificial
  Part of oligo pair for isolating Act1 transcription terminator
acaagcttggtaccgagctcactaatctctgcttttgtgcgcgt

SEQ ID NO: 11 - DNA, 24 bp, Artificial
  Part of oligo pair for isolating Act1 transcription terminator
accaatttacatggggaaaagggt

SEQ ID NO: 12 - DNA, 30 bp, Artificial
  Part of oligo pair for fusing Act1 promoter and transcription terminator
cactagtaagctgccacagcaattaatgca

SEQ ID NO: 13 - DNA, 39 bp, Artificial
  Part of oligo pair for fusing Act1 promoter and transcription terminator
tactagtggatccgatcatatgatacacggtccaatgga

SEQ ID NO: 14 - DNA, 871 bp, Artificial
  pAct1-H3-KpnI-SacI-TAct1 fragment
cactagtaagctgccacagcaattaatgcacaacatttaacctacattcttccttatcggatcctcaaaacccttaaaaacatatgcctcaccctaacatattttccaattaaccctcaatatttctctgtcacccggcctctattttccattttcttctttacccgccacgcgtttttttctttcaaatttttttcttccttcttctttttcttccacgtcctcttgcataaataaataaaccgttttgaaaccaaactcgcctctctctctcctttttgaaatatttttgggtttgtttgatcctttccttcccaatctctcttgtttaatatatattcatttatatcacgctctctttttatcttcctttttttcctctctcttgtattcttccttcccctttctactcaaaccaagaagaaaaagaaaaggtcaatctttgttaaagaataggatcttctactacatcagcttttagatttttcacgcttactgcttttttcttcccaagatcgaaaatttactgaattaacaagcttggtaccgagctcactaatctctgcttttgtgcgcgtatgtttatgtatgtacctctctctctatttctatttttaaaccaccctctcaataaaataaaaataataaagtatttttaaggaaaagacgtgtttaagcactgactttatctactttttgtacgttttcattgatataatgtgttttgtctctcccttttctacgaaaatttcaaaaattgaccaaaaaaaggaatatatatacgaaaaactattatatttatatatcatagtgttgataaaaaatgtttatccattggaccgtgtatcaggatccactagtaatcactagt

SEQ ID NO: 15 - DNA, 24 bp, Artificial
  Part of oligo pair for isolating adh1 promoter
gttgctaccagtataaatagacag

SEQ ID NO: 16 - DNA, 46 bp, Artificial
  Part of oligo pair for isolating adh1 promoter
agagctcaagcttctcgagtgtatatgagatagttgattgtatgct

SEQ ID NO: 17 - DNA, 46 bp, Artificial
  Part of oligo pai for isolating adh1 transcription terminator
cactcgagaagcttgagctctggtagatacgttgttgacacttcta

SEQ ID NO: 18 - DNA, 26 bp, Artificial
  Part of oligo pai for isolating adh1 transcription terminator
gacataaaatacacaccgagattcat

SEQ ID NO: 19 - DNA, 33 bp, Artificial
  Part of oligo pair for fusing adh1 promoter and transcription terminator
ttctagactaaaccgtggaatatttcggatatc

SEQ ID NO: 20 - DNA, 39 bp, Artificial
  Part of oligo pair for fusing adh1 promoter and transcription terminator
atctagaacgcgtgacctacaggaaagagttactcaaga

SEQ ID NO: 21 - DNA, 1003 bp, Artificial
  promoter padh1-XhoI-H3-SacI-TAdh1 (transcription terminator) fragment
gggcgaattgggcccgacgtcgcatgctcccggccgccatggccgcgggatttctagactaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacacactcgagaagcttgagctctggtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcacgcgttctagatatcactagt

SEQ ID NO: 22 - DNA, 28 bp, Artificial
  Part of oligo pair for isolating rbcL
agaattcacatgcccaagacgcaatctg

SEQ ID NO: 23 - DNA, 34 bp, Artificial
  Part of oligo pair for isolating rbcL
tctcgagccttagagcttgtccatcgtttcgaat

SEQ ID NO: 24 - DNA, 32 bp, Artificial
  Part of oligo pair for isolating rbcS
caagcttatcatgagcatgaaaactctgccca

SEQ ID NO: 25 - DNA, 32 bp, Artificial
  Part of oligo pair for isolating rbcS
agagctcaagacaaatcaggctttagtagcgg

SEQ ID NO: 26 - DNA, 35 bp, Artificial
  Part of oligo pair for isolating rpkA
actcgagatgagcaagccagatcgtgttgttttga

SEQ ID NO: 27 - DNA, 31 bp, Artificial
  Part of oligo pair for isolating rpkA
tgagctcgaaacctgagcaacctagacgcta

SEQ ID NO: 28 - DNA, 1419 bp, Synechococcus PCC6301
atgcccaagacgcaatctgccgcaggctataaggccggggtgaaggactacaaactcacctattacacccccgattacacccccaaagacactgacctgctggcggctttccgcttcagccctcagccgggtgtccctgctgacgaagctggtgcggcgatcgcggctgaatcttcgaccggtacctggaccaccgtgtggaccgacttgctgaccgacatggatcggtacaaaggcaagtgctaccacatcgagccggtgcaaggcgaagagaactcctactttgcgttcatcgcttacccgctcgacctgtttgaagaagggtcggtcaccaacatcctgacctcgatcgtcggtaacgtgtttggcttcaaagctatccgttcgctgcgtctggaagacatccgcttccccgtcgccttggtcaaaaccttccaaggtcctccccacggtatccaagtcgagcgcgacctgctgaacaagtacggccgtccgatgctgggttgcacgatcaaaccaaaactcggtctgtcggcgaaaaactacggtcgtgccgtctacgaatgtctgcgcggcggtctggacttcaccaaagacgacgaaaacatcaactcgcagccgttccaacgctggcgcgatcgcttcctgtttgtggctgatgcaatccacaaatcgcaagcagaaaccggtgaaatcaaaggtcactacctgaacgtgaccgcgccgacctgcgaagaaatgatgaaacgggctgagttcgctaaagaactcggcacgccgatcatcatgcatgacttcttgacggctggtttcaccgccaacaccaccttggcaaaatggtgccgcgacaacggcgtcctgctgcacatccaccgtgcaatgcacgcggtgatcgaccgtcagcgtaaccacgggattcacttccgtgtcttggccaagtgtttgcgtctgtccggtggtgaccacctccactccggcaccgtcgtcggcaaactggaaggcgacaaagcttcgaccttgggctttgttgacttgatgcgcgaagaccacatcgaagctgaccgcagccgtggggtcttcttcacccaagattgggcgtcgatgccgggcgtgctgccggttgcttccggtggtatccacgtgtggcacatgcccgcactggtggaaatcttcggtgatgactccgttctccagttcggtggcggcaccttgggtcacccctggggtaatgctcctggtgcaaccgcgaaccgtgttgccttggaagcttgcgtccaagctcggaacgaaggtcgcgacctctaccgtgaaggcggcgacatccttcgtgaagctggcaagtggtcgcctgaactggctgctgccctcgacctctggaaagagatcaagttcgaattcgaaacgatggacaagctctaa

SEQ ID NO: 29 - DNA, 336 bp, Synechococcus PCC6301
atgagcatgaaaactctgcccaaagagcgtcgtttcgagactttctcgtacctgcctcccctcagcgatcgccaaatcgctgcacaaatcgagtacatgatcgagcaaggcttccaccccttgatcgagttcaacgagcactcgaatccggaagagttctactggacgatgtggaagctccccctgtttgactgcaagagccctcagcaagtcctcgatgaagtgcgtgagtgccgcagcgaatacggtgattgctacatccgtgtcgctggcttcgacaacatcaagcagtgccaaaccgtgagcttcatcgttcatcgtcccggccgctactaa

SEQ ID NO: 30 - DNA, 1002 bp, Synechococcus PCC7942
atgagcaagccagatcgtgttgttctgatcggcgttgccggtgactccggttgcggcaaatcaaccttcctaaatcgccttgccgacttgtttggtacggaattgatgacggtcatctgcttggatgactatcacagtctcgatcgcaagggccggaaggaagcaggcgtaacggctttggatccccgcgccaacaactttgacttgatgtatgaacaggtcaaggcgttgaagaacggcgaaacgatcatgaagccgatctacaaccatgaaaccggcttgatcgatccgcccgaaaaaatcgaacccaatcgcatcattgtgatcgagggtctgcatccgctttacgacgagcgcgtgcgtgaactgctcgatttcagcgtttacctcgacatcgatgacgaagtcaaaatcgcttggaagatccaacgcgatatggcagaacgcggccactcctacgaagatgtcctcgcctcgatcgaagcgcgccgccctgacttcaaggcctacattgagccccagcgtggccatgcggacatcgtcatccgcgtcatgccgacccagctaatccccaatgacaccgagcgcaaggtgctgcgggtgcagttgatccaacgggaaggccgcgatggttttgagccggcttacctgttcgacgaaggttcgaccatccagtggacgccctgcggtcgtaagctgacctgctcctatccgggcattcgcttagcctacggccctggcacctactacggtcacggagtctcagtgcttgaggtcgacggtcagttcgagaacctcgaagagatgatctacgtcgagggccacctcagcaagaccgacacgcagtactacggtgagttgacccacctgctgccgcagcacaaagattacccgggttcgaacaacggcacgggtctgttccaagtgctgaccggcctgaaaatgcgggcggcctatgagcgtttgacctcccaagcagcacccgtcgccgctagcgtctag

SEQ ID NO: 31 - protein, 472 aa, Synechococcus PCC6301
MPKTQSAAGYKAGVKDYKLTYYTPDYTPKDTDLLAAFRFSPQPGVPADEAGAAIAAESSTGTWTTVWTDLLTDMDRYKGKCYHIEPVQGEENSYFAFIAYPLDLFEEGSVTNILTSIVGNVFGFKAIRSLRLEDIRFPVALVKTFQGPPHGIQVERDLLNKYGRPMLGCTIKPKLGLSAKNYGRAVYECLRGGLDFTKDDENINSQPFQRWRDRFLFVADAIHKSQAETGEIKGHYLNVTAPTCEEMMKRAEFAKELGTPIIMHDFLTAGFTANTTLAKWCRDNGVLLHIHRAMHAVIDRQRNHGIHFRVLAKCLRLSGGDHLHSGTVVGKLEGDKASTLGFVDLMREDHIEADRSRGVFFTQDWASMPGVLPVASGGIHVWHMPALVEIFGDDSVLQFGGGTLGHPWGNAPGATANRVALEACVQARNEGRDLYREGGDILREAGKWSPELAAALDLWKEIKFEFETMDKL

SEQ ID NO: 32 - protein, 111 aa, Synechococcus PCC6301
MSMKTLPKERRFETFSYLPPLSDRQIAAQIEYMIEQGFHPLIEFNEHSNPEEFYWTMWKLPLFDCKSPQQVLDEVRECRSEYGDCYIRVAGFDNIKQCQTVSFIVHRPGRY

SEQ ID NO: 33 - protein, 333 aa, Synechococcus PCC7942
MSKPDRVVLIGVAGDSGCGKSTFLNRLADLFGTELMTVICLDDYHSLDRKGRKEAGVTALDPRANNFDLMYEQVKALKNGETIMKPIYNHETGLIDPPEKIEPNRIIVIEGLHPLYDERVRELLDFSVYLDIDDEVKIAWKIQRDMAERGHSYEDVLASIEARRPDFKAYIEPQRGHADIVIRVMPTQLIPNDTERKVLRVQLIQREGRDGFEPAYLFDEGSTIQWTPCGRKLTCSYPGIRLAYGPGTYYGHGVSVLEVDGQFENLEEMIYVEGHLSKTDTQYYGELTHLLPQHKDYPGSNNGTGLFQVLTGLKMRAAYERLTSQAAPVAASV

SEQ ID NO: 34 - DNA, 1419 bp, Saccharomyces cerevisiae
atgcctaaaactcaaagtgcagcaggttataaagcaggtgtaaaagattacaaactgacttattatactcctgattatacccctaaagatactgatttgctggctgctttcaggttcagcccacagccaggagtaccagctgatgaagccggtgctgcaatcgctgccgaatccagtaccggaacatggaccacagtctggaccgatcttttaactgacatggatcgttataagggtaaatgttaccatattgaacccgttcaaggtgaagagaatagttacttcgcgtttattgcttaccctcttgatttatttgaagaaggatctgtcactaacattctaacctctatagtaggaaacgtttttggatttaaggctattaggtctttaaggttagaagatattaggtttccagtcgctctggtaaagacattccaaggtcctccccatgggatacaagtagaaagggatctattaaacaaatacggaagacctatgttgggttgtacgattaagccaaaattaggtctaagcgctaaaaactatggtcgtgcggtgtatgagtgtctaagaggtggtcttgactttacgaaagatgatgaaaacatcaattcccaaccattccaaagatggagggacagattcttattcgtggcagatgcaattcacaaaagtcaagcagaaacaggggaaatcaaaggccattatctaaacgtcaccgcgcctacatgcgaagaaatgatgaagagagccgaatttgcaaaagaattaggtactcctattattatgcatgattttctgaccgcaggttttacagccaatacaactctggctaaatggtgtagagataacggtgtgttactacacattcacagggctatgcacgccgtgatcgatagacaaagaaatcacggcatacatttcagagtcctagctaaatgtttaagattatccggtggtgatcatcttcattccggtacagttgttggaaagttggaaggtgacaaggcatctacactaggtttcgtcgacctgatgagagaagatcacatcgaagcagataggtcgagaggtgtcttttttactcaagactgggcgagtatgcctggtgttctaccagtcgcatctggtggaattcacgtttggcacatgccagctttggttgaaatatttggtgatgatagtgtattacaattcggagggggaactttgggacatccgtggggcaacgcacctggtgctaccgctaacagagttgccttagaagcttgcgttcaagctaggaacgaagggcgtgatctgtacagggaaggtggtgatattctgagggaagccggtaagtggtcacctgagttggctgccgcgttggacttatggaaagaaattaagtttgagttcgagactatggacaaactttaa

SEQ ID NO: 35 - DNA, 336 bp, Saccharomyces cerevisiae
atgtctatgaagacgctgccaaaagaaagaaggttcgaaacgttctcttacttgccacctttaagtgatagacaaatagcggcgcaaattgaatatatgatcgagcaaggcttccacccgctaattgagtttaatgaacatagtaatccagaggaattttactggactatgtggaagctgcctttgtttgattgcaagagcccacaacaagttttagatgaagttagggaatgtagatctgaatatggtgattgttacattagagtcgcaggctttgataatattaagcaatgtcagaccgtttctttcattgtccatagacccggtagatactaa

SEQ ID NO: 36 - DNA, 1002 bp, Saccharomyces cerevisiae
atgagtaaaccagacagggtagtattaattggcgtagctggtgactctgggtgcggtaaatccaccttcttgaacaggttagctgacttatttggtaccgagcttatgacggtgatctgtttagatgattaccacagcttagatagaaaaggtagaaaggaagctggcgttacagccttagatcctagagccaataacttcgatttgatgtacgaacaagtgaaagctctaaagaatggggaaaccatcatgaaaccaatttataatcacgagactggcttaatcgatccccctgagaaaattgagcccaatcgtattattgttatagaaggtttgcatccattatacgatgaaagggtcagagaattattggatttctctgtctacttggatattgatgacgaagtcaaaatagcctggaaaatccaaagggatatggctgaacgtggtcattcttatgaggacgtgcttgcctcaattgaagcacgtaggccagattttaaagcgtacattgagccacaaagaggtcatgccgatattgtgatcagagttatgccaactcagttgatccccaatgatactgaaagaaaagtcttgagagtccaattgatacaaagagagggcagagatggtttcgaacctgcttacttattcgatgaaggttcgactatacagtggactccatgtgggaggaaactaacctgctcttacccaggtattagattggcctatggtcccgacacttactatggtcatgaggtttccgttttagaagtggatggtcaattcgaaaatctagaagaaatgatttacgtagaaggacatttgtcgaagaccgatacacaatattatggggaactaactcatttactacttcagcataaagactaccctggtagtaacaatggtactggacttttccaggttctgacaggactaaagatgagagcagcctacgaaagattaacctcacaagctgcacctgttgcggcttcggtttaa

(19) United States
(12) Patent Application Publication
(10) Pub. No.: US 2008/0085341 A1
(43) Pub. Date: Apr. 10, 2008

(54) METHODS AND MICROORGANISMS FOR FORMING FERMENTATION PRODUCTS AND FIXING CARBON DIOXIDE

(75) Inventors: Ziyu Dai, Richland, WA (US); Linda L. Lasure, Fall City, WA (US); Scott E. Baker, Richland, WA (US); Jon K. Magnuson, Kennewick, WA (US)

Correspondence Address: BATTELLE MEMORIAL INSTITUTE;ATTN: IP SERVICES, K1-53, P. O. BOX 999, RICHLAND, WA, 99352, US

(73) Assignee: Battelle Memorial Institute

(21) Appl. No.: 11/846,762

(22) Filed: Aug. 29, 2007

Related U.S. Application Data
(60) Provisional application No. 60/841,722, filed on Aug. 31, 2006.

(51) Int. Cl. C12P 7/14; C12N 1/00; C12N 1/20; C13K 1/06; C12P 19/00

(52) U.S. Cl. 426/48; 435/162; 435/252.3; 435/255.1; 435/72

(57) ABSTRACT

Methods and microorganisms for forming fermentation products utilizing a microorganism having at least one heterologous gene sequence that enables carbohydrate conversion and carbon dioxide fixation in the production of the fermentation products are disclosed according to some aspects.

### STATEMENT REGARDING FEDERALLY SPONSORED RESEARCH OR DEVELOPMENT

This invention was made with Government support under Contract DE-AC0576RL01830 awarded by the U.S. Department of Energy. The Government has certain rights in the invention.

### PRIORITY

This application claims priority from U.S. provisional patent application No. 60/841,722 filed on Aug. 31, 2006.

### BACKGROUND

Fermentation has been, and continues to be, an important process for production of a variety of products in a variety of industries including food, fuel, pharmaceuticals, and other biotechnologies. Examples of products involving fermentation can include, but are not limited to a variety of typical products that are purchased and used by many consumers every day. Examples of products that rely on fermentation include various proteins and/or enzymes, antibiotics, food products, alcoholic beverages, organic acids, and fuel alcohols. In addition, a variety of base materials or precursor products, upon which other products are built, also rely upon these fermentative processes. Because of the widespread use of fermentation in chemical and product processes, improvements in fermentation efficiency can have significant impacts that cut across many industries.

In one example, the global consumption and demand for a limited liquid petroleum supply has dramatically increased in recent history. Biofuels are projected to become an increasingly significant portion of the global fuel that is consumed. Currently, bio-ethanol composes approximately 2% of the total transportation fuels mix. The necessary expansion of biofuels production within the next twenty years, in the U.S. alone, is projected to be about 30% of U.S. gasoline demand, which will equal approximately 60 billion gallons per year.

In another example, fermentation-based techniques and processes are being explored and developed for producing the building-block chemicals for polymers, which are currently derived from petroleum.

While various methodologies for producing ethanol and other fermentation products are known, there is also an ongoing search for better and more efficient ways of producing desired fermentation products. In particular, ways are sought that will produce higher yields from similar inputs, reduce waste, and improve the cost effectiveness. For at least the reasons described herein, there exists a need for improved methods and microorganisms for forming fermentation products.

### SUMMARY OF THE INVENTION

The present invention includes methods and microorganisms for forming fermentation products utilizing a microorganism having at least one heterologous gene sequence, which enables carbohydrate conversion and carbon dioxide fixation in the production of fermentation products. While a variety of embodiments of the present invention are contemplated, in a preferred embodiment of the invention, heterologous gene sequences are placed within an organism and cause the expression of exogenous enzymes that enable these organisms to fix carbon dioxide and/or convert various materials that are typically difficult to ferment, or are not fermentable by these same organisms in an unmodified state. In addition, depending upon the exact circumstances under which the invention is utilized multiple pathways, both native and exogenous, may be utilized by the organism to produce desired fermentation products. These modified organisms then can be utilized in a variety of methods whereby these exogenous enzymes enable products to be formed, modified, and processed in accordance with pre-selected criteria under a variety of conditions to achieve a desired outcome. The present invention increases the efficiency and quantity of desired fermentative products that can be produced and allows for potential increases in the number of materials that may be utilized as precursors for fermentation processing.

The purpose of the foregoing abstract is to enable the United States Patent and Trademark Office and the general public, especially the scientists, engineers, and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

Various advantages and novel features of the present invention are described herein and will become further readily apparent to those skilled in this art from the following detailed description. In the preceding and following descriptions, by way of illustration of modes contemplated for carrying out the invention, only the preferred embodiments of the invention are shown and described. As will be realized, the invention is capable of modification in various respects without departing from the invention. Accordingly, the drawings and description of the preferred embodiment set forth hereafter are to be regarded as illustrative in nature, and not as restrictive.

### DESCRIPTION OF DRAWINGS

Embodiments of the invention are described below with reference to the following accompanying drawings.

FIG. 1 is an illustration of an ethanol pathway that utilizes glucose and xylose derived from lignocellulosic or other biomasses and that fixes carbon dioxide according to embodiments of the present invention.

FIG. 2 depicts an oligo pair used for isolation of the pgk1 promoter from S. cerevisiae (SEQ ID Nos.: 1 and 2).

FIG. 3 depicts an oligo pair used for isolation of the cyc1 transcription terminator from S. cerevisiae (SEQ ID Nos.: 3 and 4).

FIG. 4 depicts an oligo pair used for fusing of the pgk1 promoter and the cyc1 transcription terminator from S. cerevisiae (SEQ ID Nos.: 5 and 6).

FIG. 5 depicts a DNA fragment containing a pgk1 promoter, EcoRI-Bc1I-Bg1II-XboI endonuclease sites, and a cyc1 transcription terminator (SEQ ID No.: 7).

FIG. 6 depicts an oligo pair used for isolation of the Act1 promoter from S. cerevisiae (SEQ ID Nos.: 8 and 9).

FIG. 7 depicts an oligo pair used for isolation of the Act1 transcription terminator from S. cerevisiae (SEQ ID Nos.: 10 and 11).

FIG. 8 depicts an oligo pair used for fusing of the Act1 promoter and transcription terminator from S. cerevisiae (SEQ ID Nos.: 12 and 13).

FIG. 9 depicts a DNA fragment containing an Act1 promoter, H3-KpnI-SacI endonuclease sites, and an Act1 transcription terminator (SEQ ID No.: 14).

FIG. 10 depicts an oligo pair used for isolation of the Adh1 promoter from S. cerevisiae (SEQ ID Nos.: 15 and 16).

FIG. 11 depicts an oligo pair used for isolation of the Adh1 transcription terminator from S. cerevisiae (SEQ ID Nos.: 17 and 18).

FIG. 12 depicts an oligo pair used for fusing of the Adh1 promoter and transcription terminator from S. cerevisiae (SEQ ID Nos.: 19 and 20).

FIG. 13 depicts a DNA fragment containing an Adh1 promoter, XhoI-H3-SacI endonuclease sites, and an Adh1 transcription terminator (SEQ ID No.: 21).

FIGS. 14a-c depict, respectively, oligo pairs used for isolation of the rbcL (SEQ ID Nos.: 22 and 23), rbcS (SEQ ID Nos.: 24 and 25), and rpkA (SEQ ID Nos.: 26 and 27) genes of synechococcus PCC6301 (rbcL & rbcS) and PCC7492 (rpkA).

FIG. 15 depicts the nucleotide coding sequence of rbcL (SEQ ID NO.: 28).

FIG. 16 depicts the nucleotide coding sequence of rbcS (SEQ ID NO.: 29).

FIG. 17 depicts the nucleotide coding sequence of rpkA (SEQ ID NO.: 30).

FIG. 18a-b depicts the protein sequence of rbcL carrying the Met259Thr mutation (SEQ ID NO.: 31).

FIG. 19 depicts the protein sequence of rbcS (SEQ ID NO.: 32).

FIG. 20 depicts the protein sequence of rpkA (SEQ ID NO.: 33).

FIG. 21 is an illustration of the expression vector pZD818 carrying the pgk1 -rbcL-Tcyc1, pAct1-rbcS-TAct1, and padh1-rpkA-Tadh1 DNA fragments.

FIG. 22 contains photographs of RNA gel-blotting analysis with a radioactively labeled probe showing transcription of rbcL, rbcS, and rpkA in glucose culture media.

FIG. 23 contains a photograph showing the results from a western blotting analysis of the RuBisCO large subunit in S. cerevisiae.

FIG. 24 contains a bar graph depicting ethanol production from engineered strains in a glucose-based medium, according to embodiments of the present invention.

FIG. 25 contains a bar graph depicting ethanol production from engineered strains in a xylose-based medium, according to embodiments of the present invention.

FIG. 26 depicts the DNA sequence of rbcL after codon usage optimization for S. Cerevisiae (SEQ ID NO.: 34).

FIG. 27a-c contains an alignment of the optimal rbcL DNA sequence (SEQ ID NO.: 34), on the lower lines, and the original rbcL sequence (SEQ ID NO.: 28), on the upper lines.

FIG. 28 depicts the DNA sequence of rbcS after codon usage optimization for S. Cerevisiae (SEQ ID NO.: 35).

FIG. 29 contains an alignment of the optimal rbcS DNA sequence (SEQ ID NO.: 35), on the lower lines, and the original rbcS sequence (SEQ ID NO.: 29) on the upper lines.

FIG. 30 depicts the DNA sequence of rpkA after codon usage optimization for S. Cerevisiae (SEQ ID NO.: 36).

FIG. 31a-b contains an alignment of the optimal rpkA DNA sequence (SEQ ID NO.: 36), on the lower lines, and the original rpkA sequence (SEQ ID NO.: 30), on the upper lines.

### DETAILED DESCRIPTION

The instant disclosure contains descriptions of various embodiments and includes the best mode of the present invention currently known. It will be clear from this description of the invention that the invention is not limited to these illustrated embodiments but that the invention also includes a variety of modifications and embodiments thereto. Therefore, the present description should be seen as illustrative and not limiting. While the invention is capable of various modifications and alternative constructions, it should be understood that there is no intent to limit the invention to the specific form disclosed, but, on the contrary, the invention is to cover all modifications, alternative constructions, and equivalents falling within the spirit and scope of the invention as defined in the claims.

As used herein, heterologous can refer to matter from, or derived from, the tissue or DNA of another species. It is typically used herein in the context of genes. Exogenous, as used herein, can refer to matter that is foreign to an organism. It is typically used herein in the context of gene products. Accordingly, for example, a heterologous DNA sequence can encode an exogenous enzyme.

Fixing, as used herein in the context of carbon dioxide, can refer to the conversion and/or incorporation of carbon dioxide, and/or the carbon atoms from carbon dioxide molecules, into organic molecules.

This application references material, such as sequence listings, found in the text file having the filename “15208-E Sequence_ST25.txt” created on Aug. 29, 2007 with a file size of 26 kb. Such material is incorporated herein by reference.

At least some aspects of the following disclosure provide methods for forming fermentation products through fermentation by modified microorganisms. In its simplest form, the methods comprise the steps of converting carbohydrates to 3-phosphoglycerate and fixing carbon dioxide, wherein various steps in the transformation of the precursor materials into the fermentative product are accomplished by enzymes expressed from heterologous genes that have been incorporated into microorganisms.

In one preferred embodiment of the invention, the converting of base materials to a pre-selected precursor such as 3-phosphoglycerate, the fixing of carbon dioxide, or both, are catalyzed by one or more exogenous photosynthetic enzymes that are produced by a microorganism in which the gene sequence for these enzymes has been introduced. In this embodiment, sugars, particularly five-carbon sugars such as xylose, form the precursor materials that are utilized by the organisms and methods of the present invention to form alcohols such as ethanol. In this preferred embodiment of the present invention the microorganisms comprise at least one heterologous gene encoding at least one enzyme, which are typically photosynthetic enzymes such as phosphoribulokinase, and one of four different forms of ribulose bisphosphate carboxylase/oxygenase (RuBisCO) (see Mueller-Cajar and Badger 2007, BioEssays 29: 722-724). While these enzymes and the pathway with which they are associated are described here in the present preferred embodiment of the invention, it is to be distinctly understood that the invention is not limited thereto but may be variously embodied and configured to include any of a variety of organisms, gene sequences, codons, enzymes, metabolites and precursor materials that would enable a party of skill in the art to achieve the contemplated ends taught by the disclosure of the present invention. Therefore while the aforementioned illustrative examples have been provided, it is to be distinctly understood that the invention is not limited thereto but may be variously embodied in accordance with the needs and necessities of a user.

The incorporation of the heterologous photosynthetic genes into the microorganism can provide the organism with the ability to enzymatically catalyze the fixation of carbon dioxide, which has previously been a substantially unutilized by-product in fermentation. This fixation of carbon dioxide can improve the productivity and efficiency of the fermentation process for the same unit of precursor material that is fed into the system. The reasons for this increased efficiency is described herebelow:

In one example, of typical ethanol fermentation, the main carbon source for the ethanol production is glucose (C₆H₁₂O₆), which is converted according to the following process:
C₆H₁₂O₆+2 ADP+2 Pi=2 C₂H₆O+2 ATP+2 CO₂ Eqn. 1
In this process, one third of the carbons from the fermentative sugar (C₆H₁₂O₆) are lost in the form of carbon dioxide. Fixation of carbon dioxide, from the fermentation process or elsewhere, provides a means of maximizing ethanol production for a given amount of fermentative sugar. Additional productivity, as described elsewhere herein, can be realized by incorporating genes conferring on the microorganism the capability to utilize sugars besides glucose including, for example, five-carbon sugars such as xylose, which can be obtained from a variety of biomasses including lignocelluloses.

Another example is lactic acid fermentation. The biochemical pathway of lactic acid fermentation is identical to ethanol production except the last reaction step, which converts the pyruvate into lactic acid by lactate dehydrogenase rather than pyruvate decarboxylase and alcohol dehydrogenase.

One advantage of the present invention is that by genetically modifying the microorganisms with exogenous enzymes, one organism may utilize a variety of types of materials in the same pathway to achieve a desired result. In addition by fixing byproducts such as carbon dioxide and reincorporating this into the system the overall system efficiency is increased. In some other embodiments of the invention the same organism may utilize multiple pathways either independently or together to achieve a desired result.

In one embodiment of the invention the exogenous photosynthetic enzymes can be expressed in a microorganism such as fungi, which can include yeasts, filamentous fungi, or both. In a specific embodiment, the yeasts can be from the genus Picchia and/or the genus Saccharomyces. Filamentous fungi, as used herein, can refer to those fungi that grow as multicellular colonies. The exogenous photosynthetic enzymes can alternatively be expressed in bacteria, archaea, or both. In a specific embodiment, the bacteria can be Zymomonas mobilis, Escherichia coli, or both.

For example, the genes of RuBisCO large subunit and small subunit from Synechococcus PCC6301, and phosphoribulokinase from Synechococcus PCC7492, were heterologously expressed in E. coli K-12 under the control of E. coli promoters and transcriptional terminators. The exogenous photosynthetic enzymes-ribulose biphosphate carboxylase/oxygenase and phosphoribulokinase from both the original and the mutated heterologous genes functioned properly in the E. coli, which incorporated the free carbon dioxide into the phosphoglycerate, an intermediate metabolite for various fermentations (see Parikh et al., 2006, Protein Engineering, Design and Selection, 19:113-119).

Suitable fermentive microorganisms are not limited by the type of sugar utilized. However, in preferred embodiments, the microorganisms can utilize six carbon sugars, such as glucose, and/or five carbon sugars, such as xylose and arabinose. In most animals and plants, as well as bacteria, yeast, and fungi, glucose is degraded initially by an anaerobic pathway prior to either oxidative or fermentative metabolism. The most common such pathway, termed glycolysis, refers to the series of enzymatic steps whereby the six-carbon glucose molecule is broken down, via multiple intermediates, into two molecules of the three carbon compound, pyruvate. During this process, two molecules of NAD+ are reduced to form NADH. The net reaction in this transformation of glucose into pyruvate is:
Glucose+2 Pᵢ+2 ADP+2 NAD⁺→2 pyruvate+2 ATP+2 NADH+2 H⁺ Eqn. 2
For glycolysis to continue, the NAD+ consumed by glycolysis must be regenerated by the oxidation of NADH. During oxidative metabolism, NADH typically is oxidized by donating hydrogen equivalents via a series of steps to oxygen, thereby liberating free energy to form ATP and forming water. Most organisms contain additional anaerobic pathways, however, which allow glycolysis to continue in the absence of compounds like oxygen (i.e., fermentation). Carbon dioxide fixation in these additional pathways can be utilized according to embodiments of the present invention to improve the yield of the metabolites and/or fermentation products associated with the additional pathways.

Five carbon sugars, which can also be converted to ethanol, are abundant in nature as a major component of lignocellulosic biomass. One such five carbon sugar is xylose, which is second only to glucose in natural abundance. As with six carbon sugars, five carbon sugars can be converted into various fermentation products through the appropriate pathways. In one approach, xylose can be converted into pyruvate by modified glycolytic pathways. The pyruvate can then be redirected to ethanol. In another approach, xylose can be be converted to xylulose using xylose isomerase prior to fermentation by S. cerevisiae. The typical net reaction for a five carbon sugar, wherein three pentose sugars yield five ethanol and five carbon dioxide molecules, can also be improved by fixation of carbon dioxide, as described in the following examples as well as elsewhere herein.

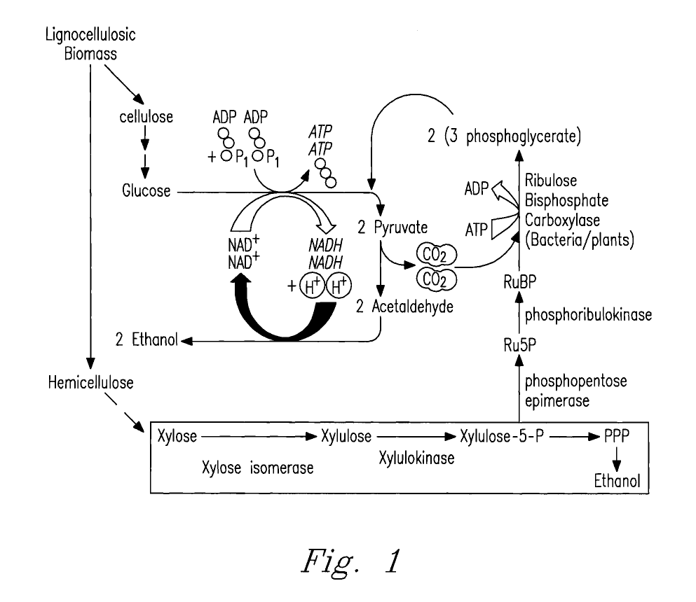

Referring to FIG. 1, an illustration is shown of an ethanol fermentation pathway from organisms capable of alcoholic glucose fermentation. The traditional glucose pathway through pyruvate and acetaldehyde is supplemented by an engineered pathway for converting xylose to xylulose and then to ethanol through the pentose phosphate pathway. Ethanol production from xylose has been reported in a variety of microorganisms and is known in the art. For instance, details regarding the conversion of xylose to ethanol have been described for yeasts as well as for Z. mobilis and other bacteria.

According to the illustration and to one embodiment of the present invention, the ethanol productivity resulting from either, or both, of the known pathways can be improved by introducing an additional engineered pathway that fixes carbon dioxide. Specifically, in the native or engineered xylose-utilizing organisms, xylose would typically be converted to D-xylulose-5-phosphate by xylose isomerase and xylulokinase or by xylose reductase, xylitol dehydrogenase and xylulokinase. It can be further isomerized to ribulose-5-phosphate in the pentose phosphate pathway (PPP), which can then be converted to ribulose-1,5-bisphosphate by phosphoribulokinase. The two conversions are catalyzed, respectively, by the endogenous ribulose-5-phosphate epimerase and exogenous phosphoribulokinase, a photosynthetic enzyme. In the microorganisms that do not utilize xylose, the carbon dioxide fixation pathway is still active and uses ribulose-5-phosphate generated through the pentose phosphate pathway, through which a portion of the available glucose is metabolized, to form the ribulose-1,5-bisphosphate mediated by the exogenous phosphoribulokinase.

Fixation of carbon dioxide can then be catalyzed by ribulose 1,5-bisphophate carboxylase (i.e., RuBisCO) by converting the ribulose 1,5-bisphosphate and carbon dioxide into 3-phosphoglycerate, which is an intermediate metabolite for ethanol production.

Phosphopentose epimerase, the enzyme that catalyzes the conversion of xylulose-5-phosphate to ribulose-5-phosphate, exists ubiquitously in all living cells including the microorganisms, such as yeasts that are within the scope of the present invention. Phosphoribulokinase and RuBisCO are exogenous photosynthetic enzymes expressed from heterologous genes integrated in the microorganisms encompassed by embodiments of the present invention. Accordingly, fermentive microorganisms that utilize five and/or six carbon sugars, whether they are engineered or are wild-type, can be modified and/or utilized according to embodiments of the present invention for improved production of ethanol and fixation of carbon dioxide.

The transformation of yeast using plasmids that contain genes encoding photosynthetic enzymes for fixing carbon dioxide is described below as an example of a transgenic yeast strain capable of forming fermentation products from sugars and fixing carbon dioxide. Additional examples are provided describing the characterization and performance results of the transgenic yeast strain.

### EXAMPLE

### Incorporation into Yeast of Genes Encoding Photosynthetic Enzymes

Carbon dioxide-fixing S. cerevisiae were developed by transformation of the S. cerevisiae strain YVH10 using plasmid pZD818. The plasmid comprised genes for ribulose-1,5-bisphosphate carboxylase large (rbcL) and small (rbcS) subunits and for phosphoribulokinase (rpkA) were isolated from cyanobacteria of the synechococcus PCC6301 (rbcL & rbcS) and PCC7492 (rpkA), respectively.

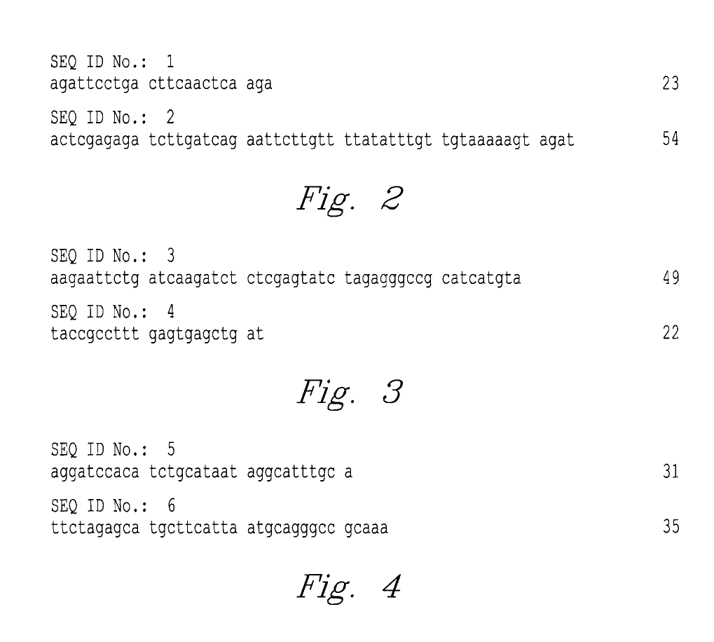

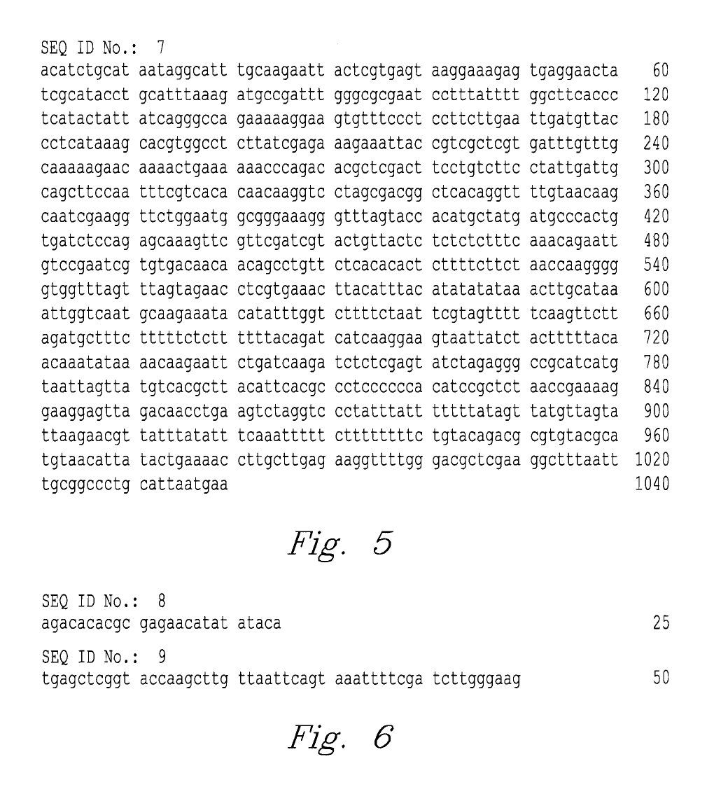

The phosphoglycerate kinase (pgk1) promoter and the iso-1-cytochrome C (cyc1) transcription terminator were isolated from S. cerevisiae. The oligo pair (SEQ ID NOs.: 1 and 2) shown in FIG. 2 was used to isolate the pgk1 promoter. The oligo pair (SEQ ID NOs.: 3 and 4) shown in FIG. 3 was used to isolate the cyc1 transcription terminator. The pgk1 promoter and the cyc1 transcription terminator were separately isolated via genome polymerase chain reaction (PCR) and then fused together via overlap PCR with the pair of oligos (SEQ ID NOs.: 5 and 6) shown in FIG. 4. Several restriction endonuclease sites (EcoRI-Bc1I-Bg1II-XboI) were introduced via PCR. The combined promoter (pgk1)/restriction endonuclease sites (EcoRI-Bc1I-Bg1II-XboI)/transcription terminator (Tcyc1) fragment was cloned into a pGEM-T vector and the resultant nucleotide sequence of this combined fragment (SEQ ID NO.: 7) is shown in FIG. 5.

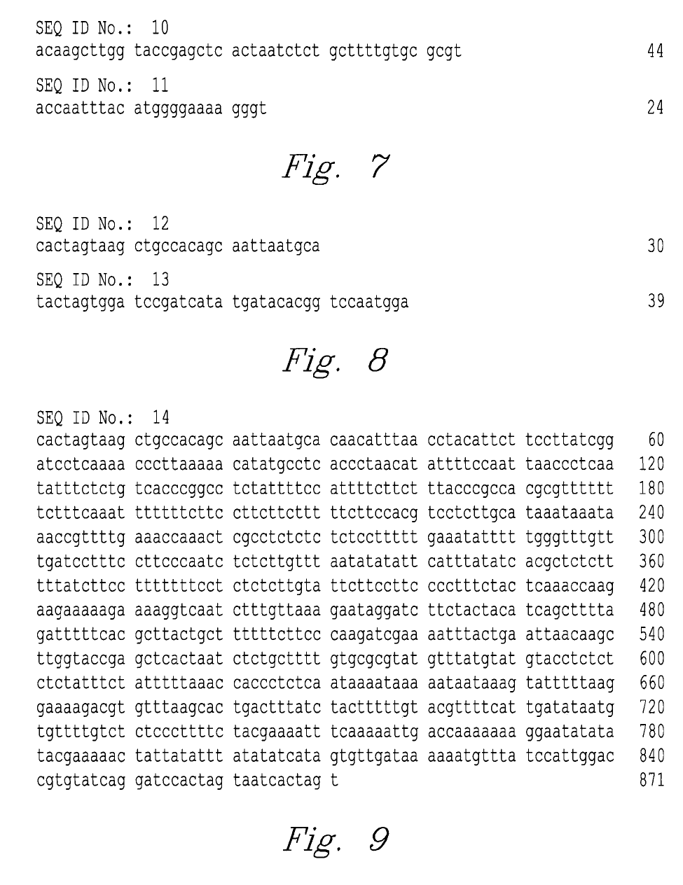

The actin (Act1) promoter and its transcription terminator were isolated from S. cerevisiae. The oligo pair (SEQ ID NOs.: 8 and 9) shown in FIG. 6 was used to isolate the Act1 promoter. The oligo pair (SEQ ID NOs.: 10 and 11) shown in FIG. 7 was used to isolate the Act1 transcription terminator. The Act1 promoter and its transcription terminator were separately isolated via genome PCR and then fused together via overlap PCR with the pair of oligos (SEQ ID NOs.: 12 and 13) shown in FIG. 8. Several restriction endonuclease sites (H3-KpnI-SacI) were introduced via PCR. The combined promoter (Act1)/restriction endonuclease sites (H3-KpnI-SacI)/transcription terminator (TAct1) fragment was cloned into a pGEM-T vector and the resultant nucleotide sequence of this combined fragment (SEQ ID NO.: 14) is shown in FIG. 9.

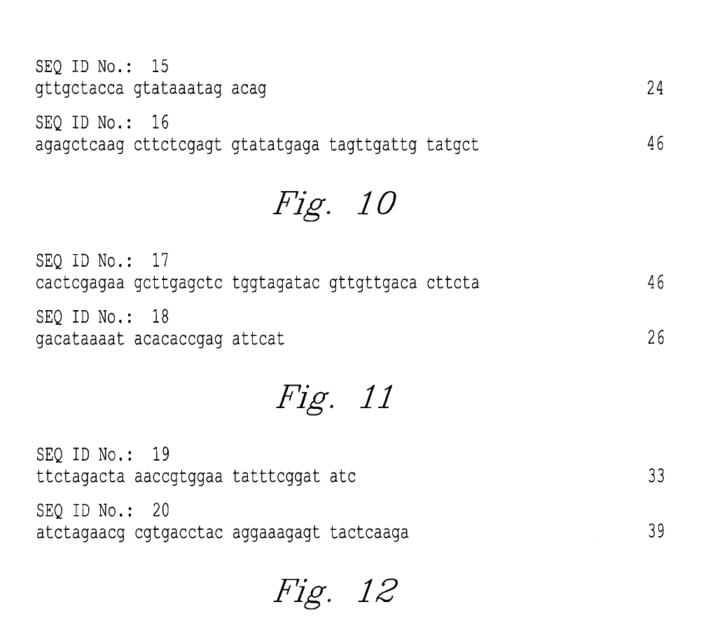

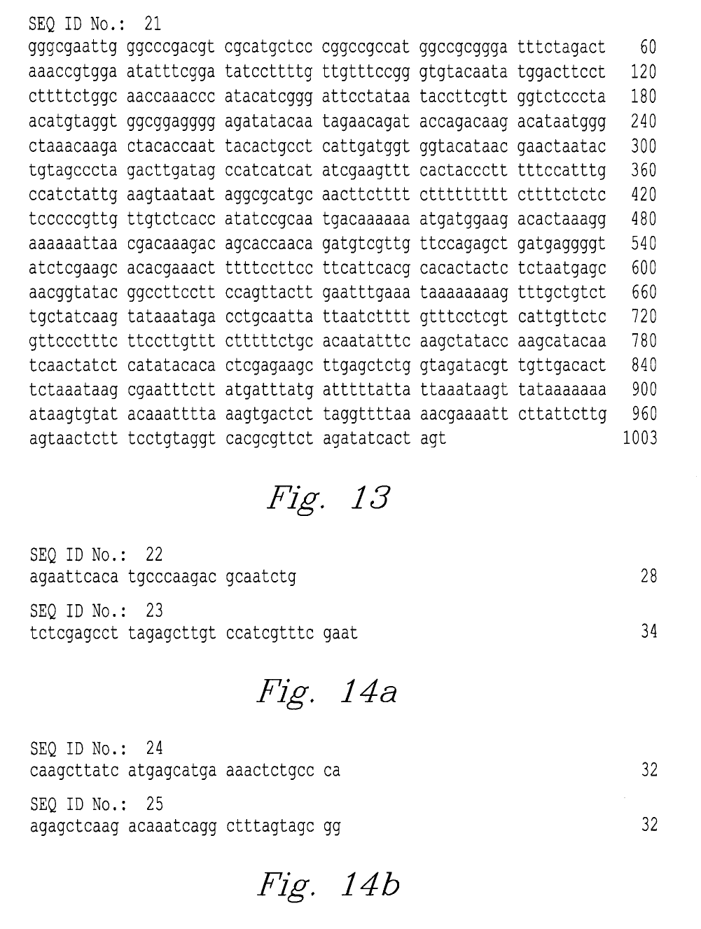

The alcohol dehydrogenase (adh1) promoter and transcription terminator were isolated from S. cerevisiae. The oligo pair (SEQ ID NOs.: 15 and 16) shown in FIG. 10 was used to isolate the adh1 promoter. The oligo pair (SEQ ID NOs.: 17 and 18) shown in FIG. 11 was used to isolate the adh1 transcription terminator. The adh1 promoter and its transcription terminator were separately isolated via genome PCR and then fused together via overlap PCR with the pair of oligos (SEQ ID NOs.: 19 and 20) shown in FIG. 12. Several restriction endonuclease sites (XhoI-H3-SacI) were introduced via PCR. The combined promoter (adh1)/restriction endonuclease sites (XhoI-H3-SaI)/transcription terminator (Tadh1) fragment was cloned into a pGEM-T vector and the resultant nucleotide sequence of this combined fragment (SEQ ID NO.: 21) is shown in FIG. 13.

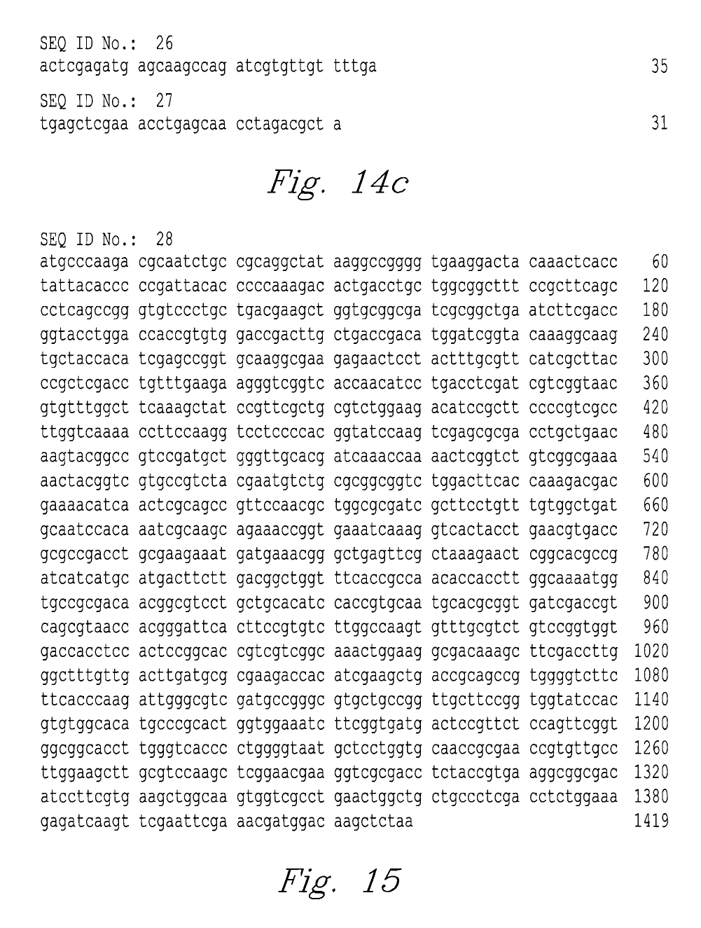

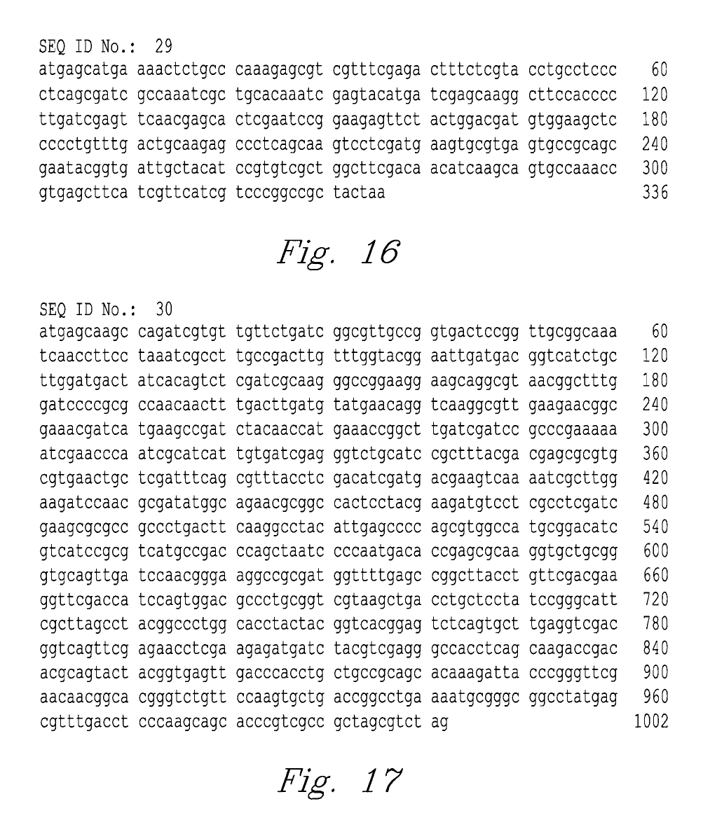

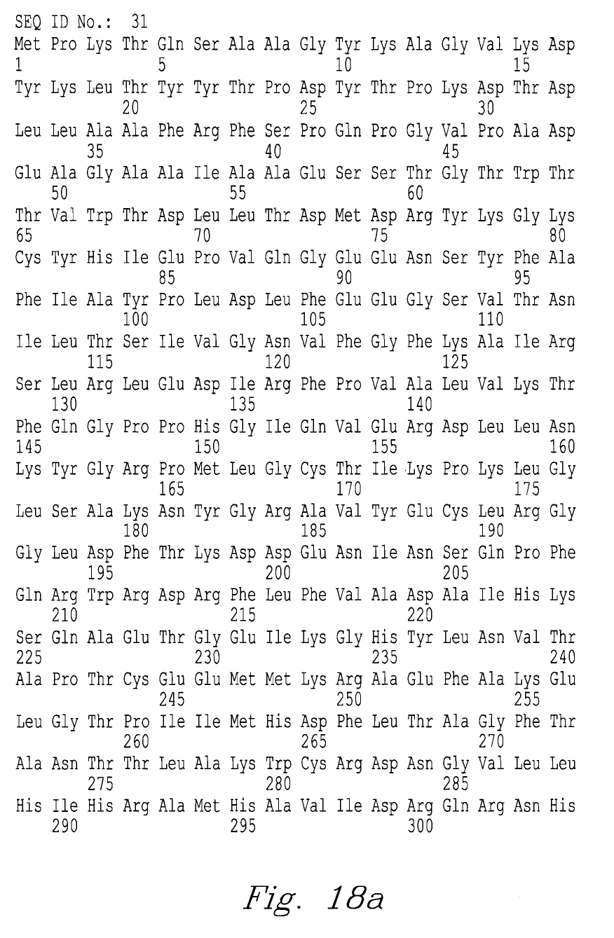

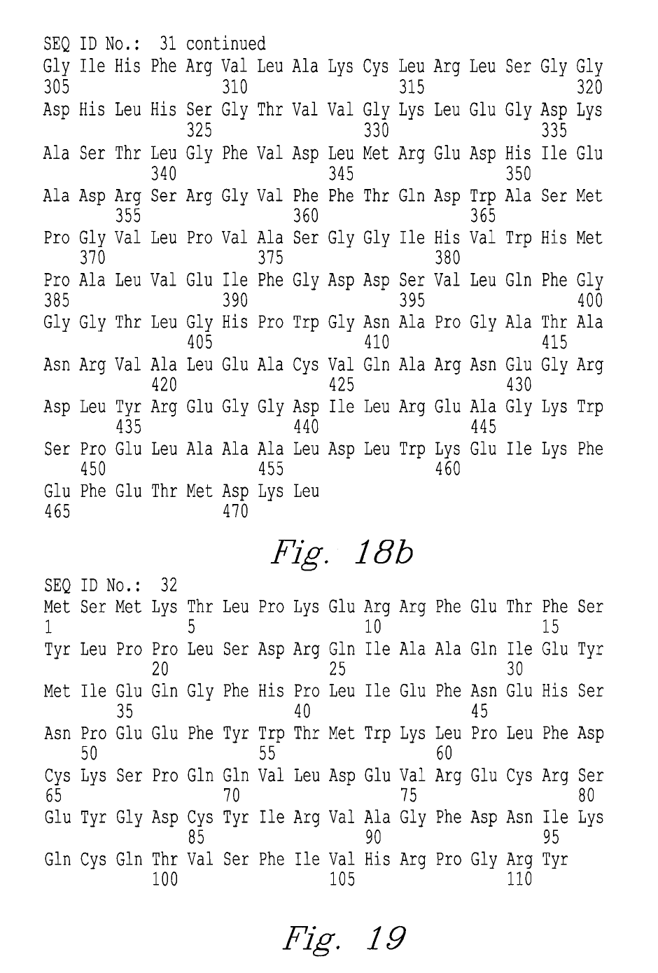

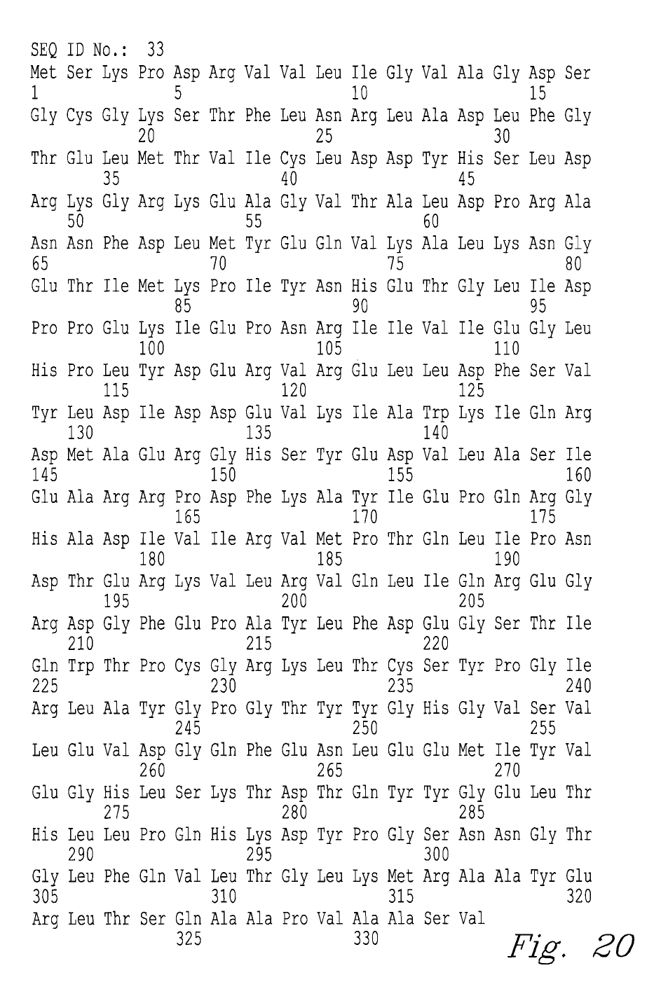

Three genes that encode ribulose-1,5-bisphosphate carboxylase large subunit (rbcL) with the Met259Thr mutation, ribulose-1,5-bisphosphate carboxylase small subunit (rbcS), and phosphoribulokinase (rpkA), which contribute to carbon fixation, were isolated from plasmid DNA rbcLS-pET30a+(mutant 2.29; for rbcL and rbcS) and P_{BAD}-6His-prkA-pACYC 184 (for rpkA) by PCR. The rbcL, rbcS and rpkA were originally isolated from the cyanobacteria Synechococcus PCC6301 (rbcL & rbcS) and PCC7492 (rpkA), respectively. Oligo pairs, which are shown in FIGS. 14a-14c, were designed and synthesized for isolation of rbcL, rbcS, and rpkA, respectively. The oligo pairs for isolating rbcL, rbcS, and rpkA are listed in SEQ ID NOs.: 22-23, 24-25, and 26-27, respectively, where the oligo pairs contain proper restriction endonucleases sites at the location prior the translation start-codon ATG and stop-codons (TAA or TAG) for further cloning. Plasmid DNA of rbcLS-pET30a+ with the T776C mutation and P_{BAD}-6His-prkA-pACYC184/K-12 were obtained from the Department of Biochemistry, Center for Fundamental and Applied Molecular Evolution at the Emory University School of Medicine. Details regarding the plasmid DNA are described by Parikh et al., in Protein Engineering, Design, and Selection, vol. 19(3), pp. 113-119, 2006, which details are incorporated herein by reference. The plasmid DNA was used as a template for PCR isolation of the genes for rbcL, rbcS, and rpkA. The PCR products were then cloned into the pGEM-T vector and confirmed by DNA sequencing. The nucleotide coding sequences of rbcL (SEQ ID NO.: 28), rbcS (SEQ ID NO.: 29), and rpkA (SEQ ID NO.: 30) are shown in FIGS. 15, 16, and 17, respectively. Their corresponding protein sequences (SEQ ID NOs.: 31, 32, and 33, respectively) are shown in FIGS. 18, 19, and 20, respectively. The sequence-confirmed DNA fragment of the rbcL gene was in-frame fused with promoter pgk1 and transcription terminator cyc1. The sequence-confirmed DNA fragment of the rbcS gene was in-frame fused with promoter Act1 and transcription terminator Act1. The sequence-confirmed DNA fragment of the rpkA gene was in-frame fused with promoter adh1 and transcription terminator adh1.

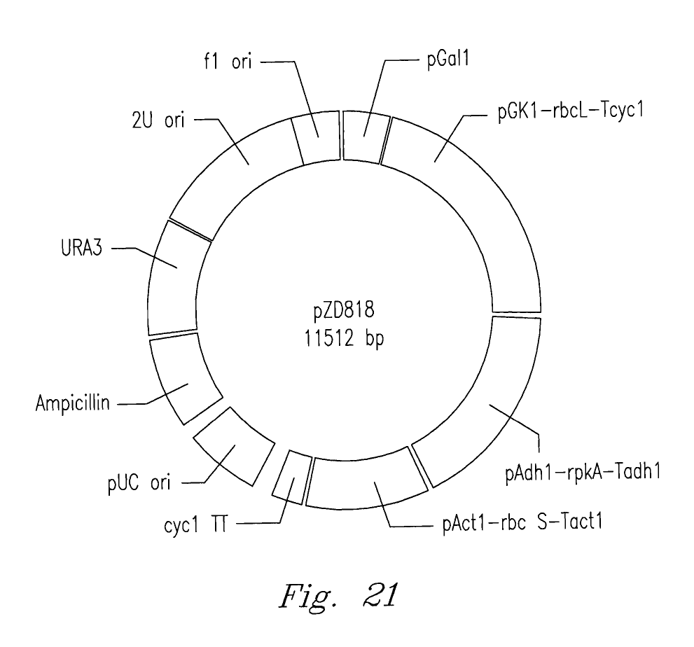

The yeast expression vectors were constructed on the basis of the pYES2 plasmid vector. Referring to FIG. 21, the expression vector pZD818 was formed by excising the DNA fragments of pgk1-rbcL-Tcyc1, pAct1-rbcS-TAct1, and padh1-rpkA-Tadh1 with the restriction endonuclease NcoI/NdeI, NcoI/Ndel and BamHI, respectively, treating the fragments with DNA polymerase I (large Klenow fragment), and sequentially inserting them into the pYES2 plasmid vector at the restriction endonuclease site HindIII, BamHI, and EcoRI, respectively, which were also treated with DNA polymerase I (large Klenow fragment) sequentially.

Transformation of yeast cells with the expression vector pZD818 was carried out according to methods described by Gietz et al. (Gietz et al., Yeast, vol. 11, pp. 355-360, 1995), which details are incorporated herein by reference. The plasmid DNA was transferred into S. cerevisiae strain YVH10 (Ura-, Trip-), which is derived from parent strain BJ5464 and is MAT-α, that was selected on a selected growth media (SD-CAA+Trp). The SD-CAA medium comprised 5.0 g/l ammonium sulfate, 1 ml 1000×vitamins, 1 ml 1000×trace elements, 1.0 g/l KH₂PO₄, 0.5 g/l MgSO₄, 0.1 g/l NaCl, 0.1 g/l CaCl₂, 100 g/l D-glucose, and 0.5 g/l synthetic complete dropout mix. The dropout mix comprised two grams of each amino acid of adenine hemisulfate, arginine HCl, histidine HCl, isoleucine, leucine, lysine HCl, methionine, phenylalanine, and Tyrosine, as well as 6 grams homoserine, 3 grams tryptophane, and 9 grams of valine. Briefly, the method involves:
a) growing the YVH10 in 50 ml SD-CAA medium to the cell density of 4×10⁷ cells/ml culture;
b) centrifuging to remove the SD-CAA medium and resuspending the cells into 1 ml 100 mM lithium acetate (LiAc);
c) centrifuging again to remove the LiAc and finally re-suspending the cells with PEG-LiAc transformation mixture with the plasmid DNA pZD818; and
d) centrifuging to remove the transformation mixture and growing the cells on a selected growth media (SD-CAA +Trp) plate at 30° C. for 2-4 days.

The transformed yeast colonies were randomly chosen for further characterization, the results of which are described elsewhere herein. The transgenic S. cerevisiae strains derived from YVH10 (Ura-, Trp-), which carried the heterologous photosynthetic genes cloned into expression vector pYES2, were named TY-3730, TY-3731, TY-3732 and TY3733. A transgenic control strain was derived from YVH10 (Ura-, Trp-) with only the empty pYES2 vector and was named yES2C.

### EXAMPLE

### RNA-Blotting Analysis of Transformed Yeast Cells

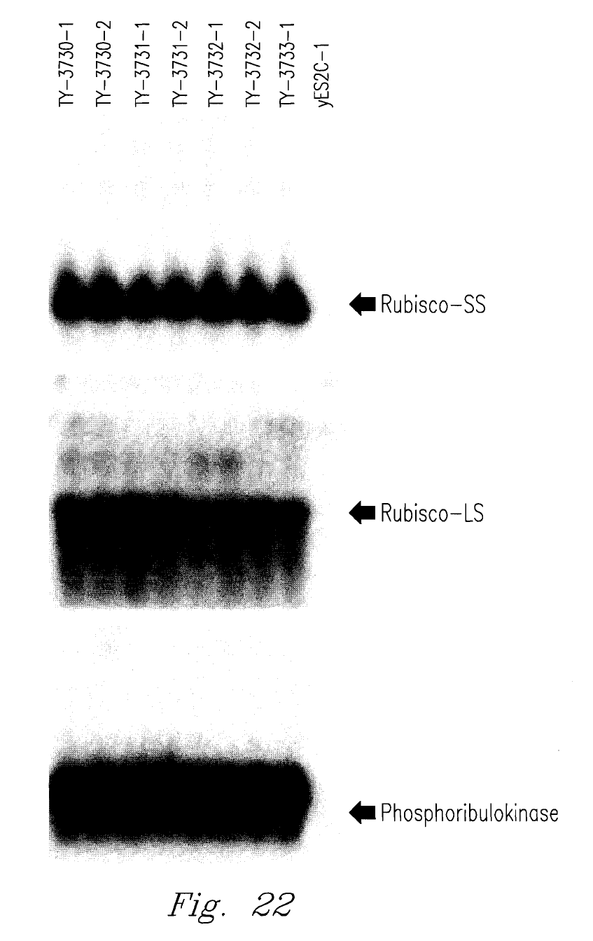

The transgenic yeast strains, TY-3730, TY-3731, TY-3732, TY3733, and yES2C, were grown in a yeast selected medium (SD-CAA) at 30° C. and 130 rpm for approximately 20 hours. The total RNA of the yeast cells was isolated using a kit for total RNA isolation sold under the tradename, RNEASY Mini Kit, which is available from QIAGEN in Valencia, Calif., USA. Using the RNEASY Mini Kit, samples are first lysed and then homogenized. Ethanol is added to the lysate to provide ideal binding conditions. The lysate is then loaded onto the RNEASY silica-gel membrane. RNA binds, and all contaminants are efficiently washed away. Pure, concentrated RNA is eluted in 10 mM Tris-HCl buffer. In the instant example, twenty micrograms of total RNA per sample were separated via formaldehyde gel electrophoresis and then transferred onto a Zeta-probe blotting membrane. Method for the RNA-blotting analysis was described by Dai et al. (2004, Applied Environmental Microbiology, 70: 2474-2485). The RNA-blotting analysis results are provided in FIG. 22 and show that all three heterologous photosynthetic genes for rbcL, rbcS, and rpkA had high transcription levels in the glucose culture selected medium.

### EXAMPLE

### Cell Extract for ¹⁴CO₂ Incorporation Assay and Western Blotting Analysis

The transgenic yeast strains, TY-3730, TY-3731, TY-3732, TY-3733, and yES2C, were grown in a yeast selected medium (SC-CAA+Trp) at 30° C. and 130 rpm for approximately 20 hours. Propagated transformed cells were harvested by centrifugation. The yeast cells were then re-suspended in a mixture comprising a TEM buffer (925 mM Tris-HCl, pH 8.0, 2 mM EDTA, and 10 mM β-mercaptoethanol) and a 10 μl/ml Protease Inhibitor Cocktail (P8215 from Sigma-Aldrich, St. Louis, Mo.) to wash out the culture medium and for re-centrifugation. The yeast cell pellets were immediately frozen in liquid nitrogen. For cell extraction, the frozen cell pellets were ground to a fine powder using a frozen mortar and pestle. Five hundred microliters of a RubisCO assay buffer (50 mM Bicine, pH 8.0, 20 mM MgCl₂, 10 mM NaHCO₃, and 10 mM β-mercaptoethanol) and 10 μl/ml of the protease inhibitor cocktail were added into the mortar before the cell mass was thawed. Grinding continued until the cell mass had thawed. The cell extract was then transferred into a microcentrifuge tube and centrifuged at 10,000 g. The supernatant was transferred into a new microcentrifuge tube for ¹⁴CO₂ incorporation assay and western blotting analysis.

For the ¹⁴CO₂ incorporation assay, 160 μl of assay medium comprising 100 mM bicine (pH 8), 1 mM EDTA, 30 mM MgCl₂, 20 mM NaH¹⁴CO₃ 5mM DTT, and 0.5 mM ribulose 1,5-biphosphate was first incubated in a water bath at 30° C. for 5 min. Forty microliters of cell extract were added into the assay medium and mixed well to initiate the ¹⁴CO₂ incorporation reaction. The reaction was quenched after 1 min by the addition of 100 μl of 1 M HCl. The acidified reaction mixtures were incubated at 70° C. in a fume hood to evaporate the unincorporated ¹⁴CO₂. The acid-stable, incorporated ¹⁴CO₂ was dissolved in 100 μl de-ionized H₂O and its radioactivity was measured in a scintillation counter after mixing well with 10 ml scintillation liquid. The amount of ¹⁴CO₂ incorporation in potato leaf cells, yES2C control cells, TY-3730 cells, and TY-3732 cells is shown for comparison in Table 1. The results indicate that the transgenic yeast strains exhibit improved ¹⁴CO₂ incorporation relative to the control sample.

TABLE 1
Summary of the ¹⁴CO₂ incorporation in various samples

| Potato Leaves | yES2C-1 | TY-3730-1 | TY-3732-1 |
|---|---|---|---|
| 3405 cpm | 175 cpm | 328 cpm | 258 cpm |

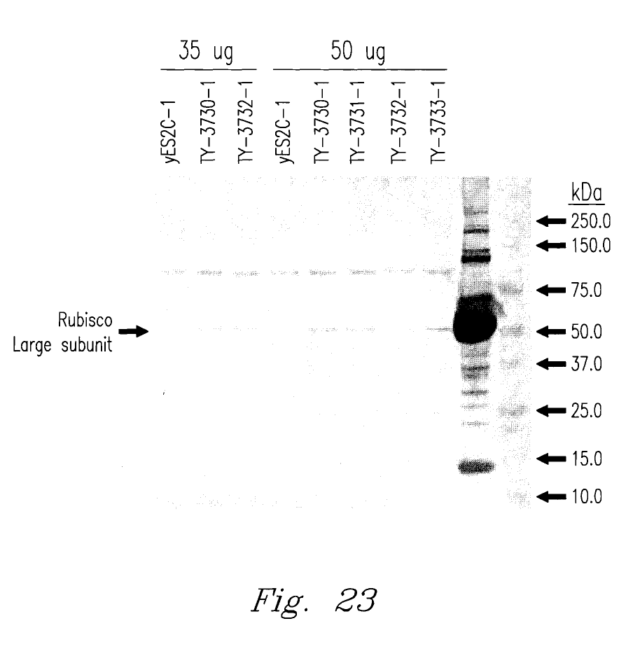

For the western blotting analysis, 35 to 50 μg of total soluble proteins from the cell extracts of the potato leaves and the transgenic strains used for the ¹⁴CO₂ incorporation assay were analyzed using a 5 to 15% gradient sodium dodecyl sulfate polyacrylamide gel electrophoresis (SDS-PAGE). The total proteins in the gel were electrophoretically transferred onto a nitrocellulose membrane. The nitrocellulose membrane was first blocked with blocking solution comprising 5% nonfat dry milk in a TTBS buffer, which contained 20 mM Tris-HCl, 150 mM NaCl, and 0.05% Tween 20, having a pH value of 7.5. The nitrocellulose membrane was then incubated with the rabbit polyclonal antibody against soybean RuBisCO large-subunit in TTBS solution. Finally, the membrane was blotted with anti-rabbit IgG alkaline phosphatase. The RuBisCO large-subunit proteins on the membrane were visualized by color development. The results of the SDS-PAGE gel are shown in FIG. 23 and show that the RuBisCO large-subunit was indeed accumulated in S. cerevisiae properly.

Examination of fermentation by the transgenic yeast strains TY-3730, TY-3731, TY-3732, and TY-3733 indicated that ethanol production improved, relative to the control strain, for glucose as well as for xylose.

### EXAMPLE

### Ethanol Production in Transgenic Yeast Grown in a Glucose-Based Selected Growth Medium

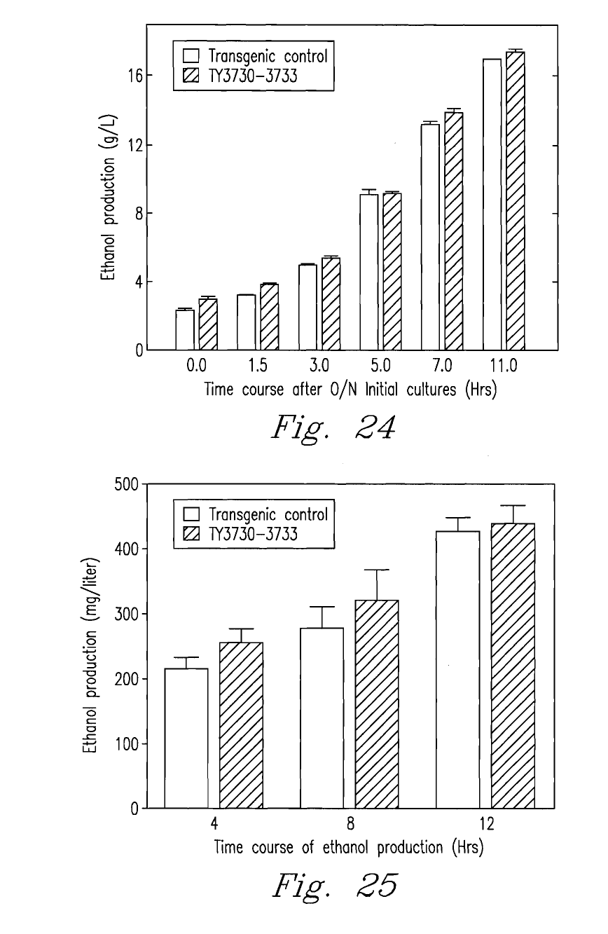

Characterization of ethanol production in transgenic yeast grown in glucose-based selected growth medium is described below. The transgenic S. cerevisiae strains TY-7330, 7331, 7332, 7333 and transgenic control yES2C were first grown in 3 ml SD-CAA+Trp medium with 100 g/l D-glucose as carbon source in the culture tubes for about 16 hrs to have enough cell density for ethanol fermentation. The total cells in each culture were determined spectrophotometrically. Equal cell densities were used for further flask cultures of 50 ml with the same culture medium. The cultures were sampled every three hours for ethanol content and cell density. The results are shown in FIG. 24. At least 12 independent culture repeats were performed for each time point in the figure. The average ethanol production in transgenic S. cerevisiae strains TY-7330, 7331, 7332 and 7333 was about 2 to 25% higher than the transgenic control strains.

### EXAMPLE

### Ethanol Production in Transgenic Yeast Grown in a Xylose-Based Selected Growth Medium

Characterization of ethanol production in transgenic yeast grown in xylose-based selected growth medium containing glucose isomerase is described below. The transgenic S. cerevisiae strains TY-7330, 7331, 7332, 7333 and transgenic control yES2C were first grown in a SD-CAA (-uracil, -tryptophane) medium supplemented with 40 mg/l Tryptophane for about 20 hrs to have enough cell density for ethanol fermentation. The total cells in each culture were determined spectrophotometrically. Equal cell densities were used for further flask cultures of 50 ml SD-CAA+Trp medium with 100 g/l D-xylose as carbon base. The cultures were sampled every three hours for measuring ethanol content and cell densities. The ethanol content was determined enzymatically. The results are shown in FIG. 25. At least 12 independent culture repeats were performed for each time point in the figure. The improvement of ethanol production in transgenic yeast strains TY-7330, TY-7331, TY-7332, and TY-7333 was about 3% to 20% compared to the transgenic control strains (yES2C).

### EXAMPLE

### Codon Optimization in Transgenic S. cerevisiae

The RNA gel-blotting analyses described above showed that the rbcL, rbcS, and rpkA can be highly expressed in S. cerevisiae. However, the level of protein accumulation was relatively low. To improve the total protein amounts of rbcL, rbcS, and rpkA in the transgenic yeast, the yeast's codon usage bias can be optimized. Codons are triplets of nucleotides that together specify an amino acid residue in a polypeptide chain. There are 64 possible triplets to recognize only 20 amino acids plus the translation termination signal. Because of this redundancy, all but two amino acids are coded for by more than one triplet. Different organisms often show particular preferences for one of the several codons that encode the same given amino acid. It has been demonstrated that optimal codons can help to achieve faster translation rates and higher accuracy (see Man and Pilpel, 2007, Nature Genet, 39: 415-421; and Kliman et al., 2003, J. Molecular Evolution, 57: 98-109). Accordingly, the exogenous protein expression in the transgenic S. cerevisiae can be improved via codon usage optimization and the protein expression levels of the de-novo genes may thereby be substantially increased in the transgenic S. cerevisiae.

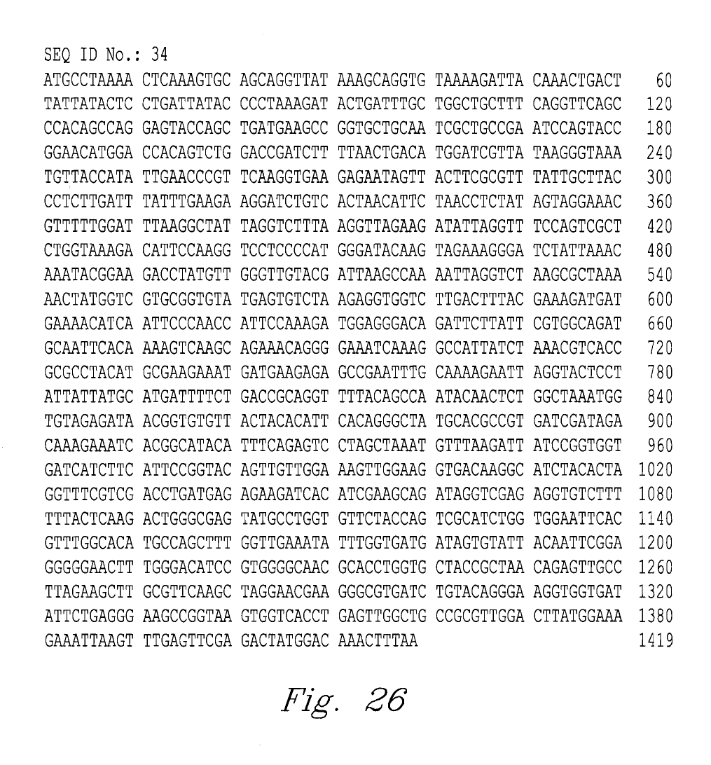

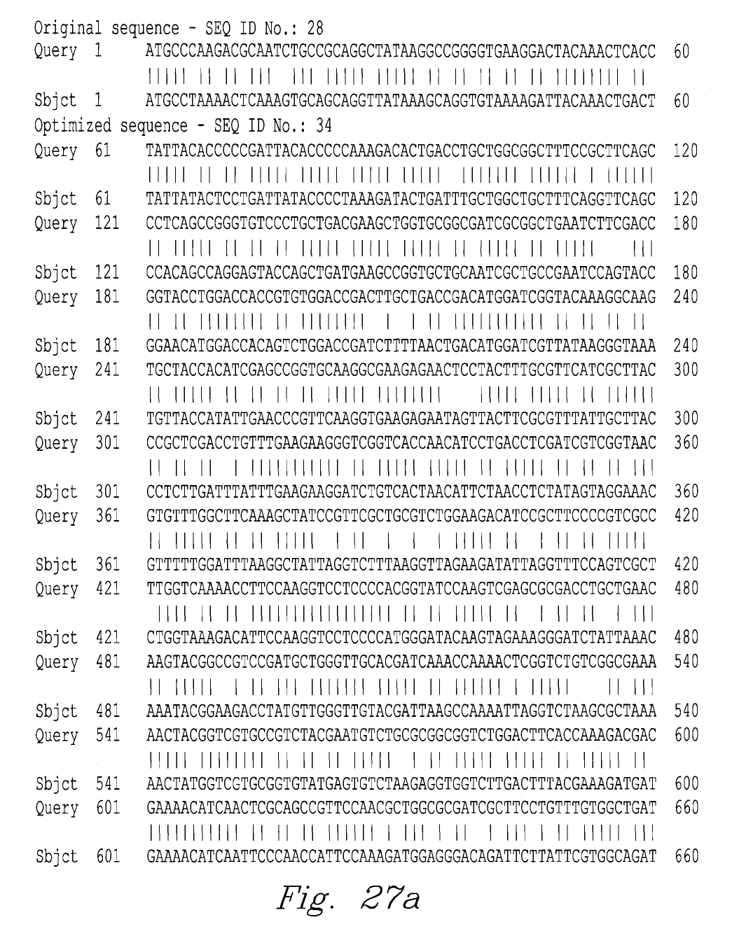

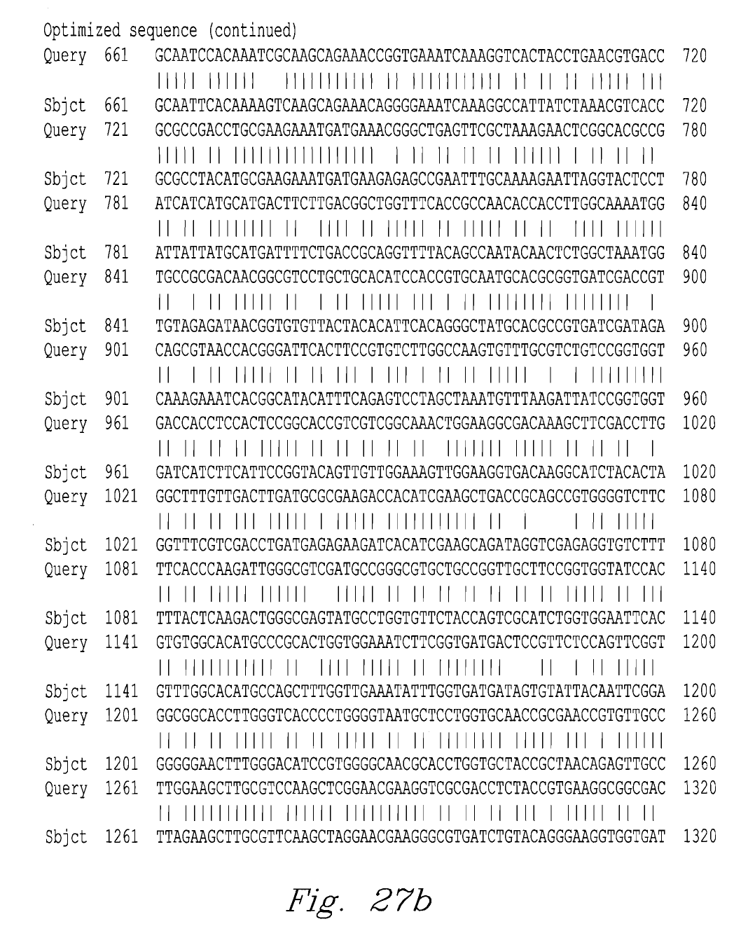

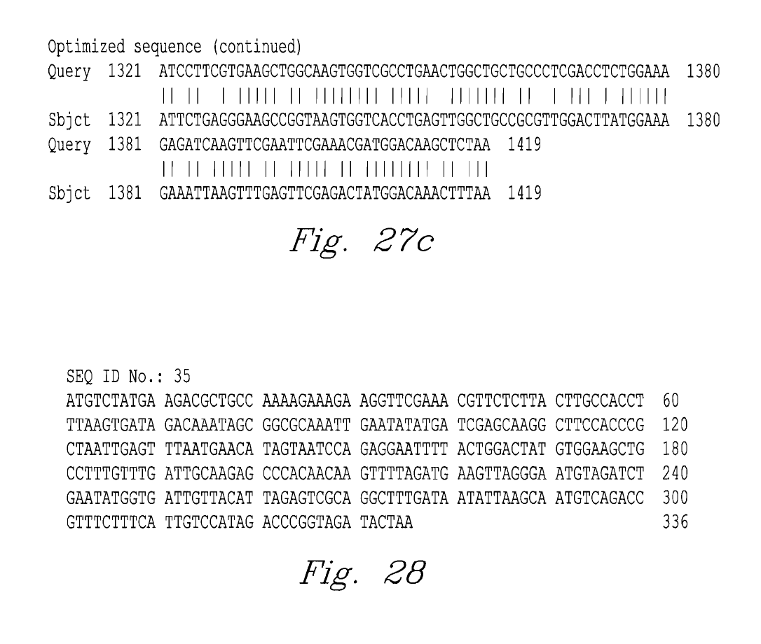

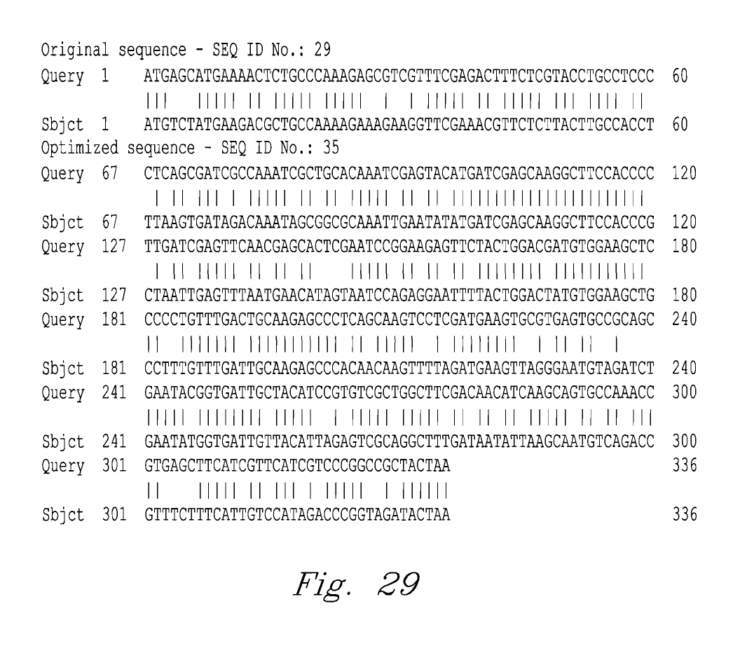

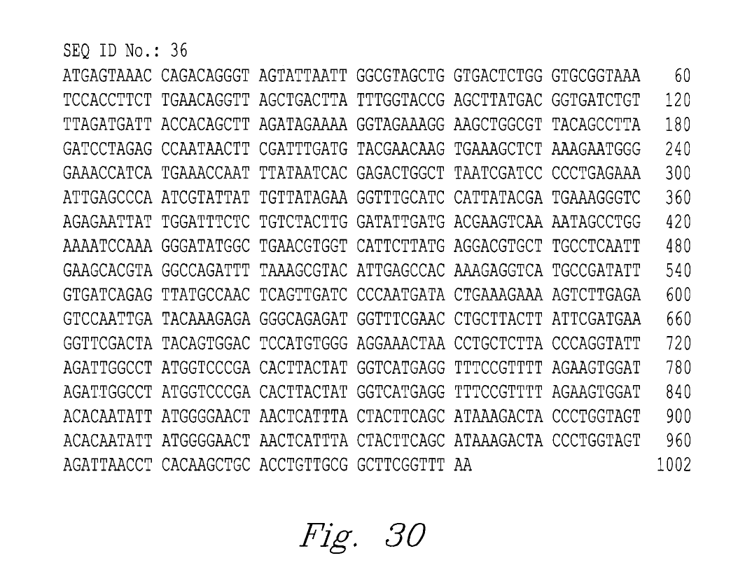

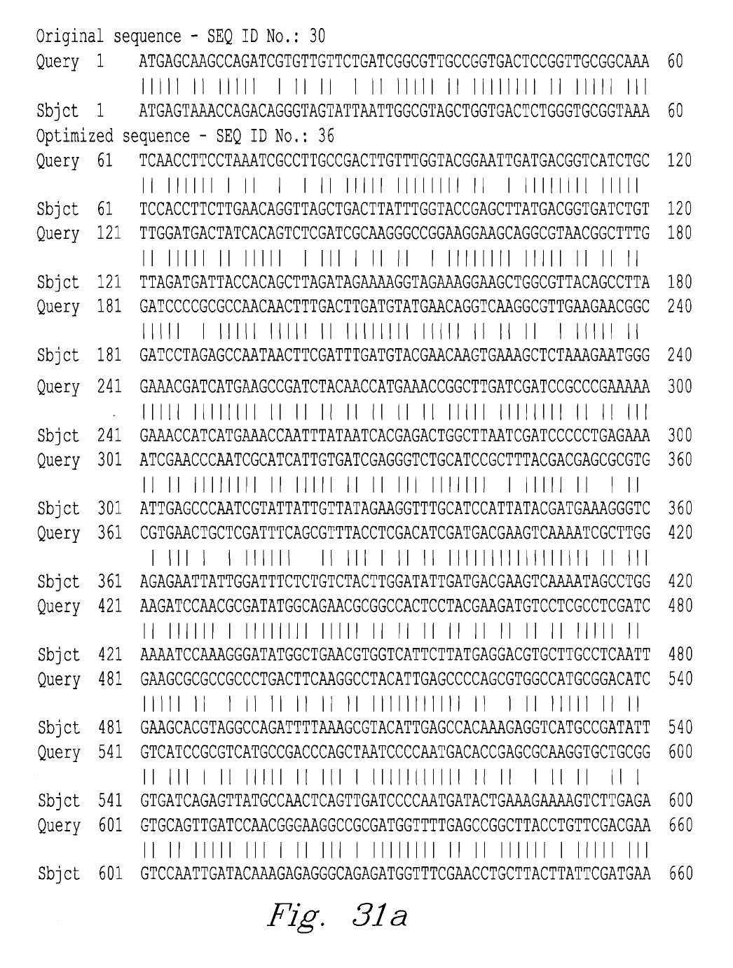

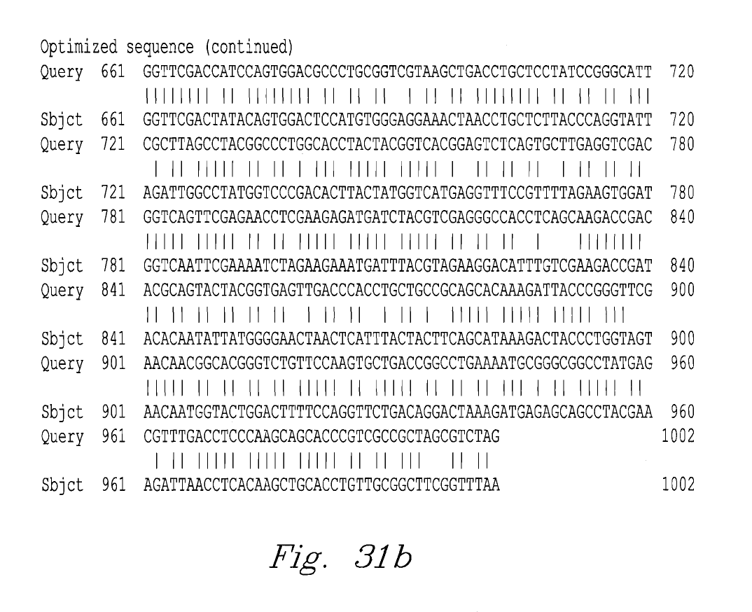

The codon usage optimization of the present embodiment was mainly based on the codon usage database of S. cerevisiae in the public database and on the GENE DESIGNER software by DNA2.0, Inc. (Menlo Park, Calif., USA), which is a design tool for molecular biologists that allows one to optimize expression by codon optimizing proteins for any expression host. The DNA sequences of rbcL (SEQ ID NO.: 34), rbcS (SEQ ID NO.: 35), and rpkA (SEQ ID NO.:36) after the codon usage optimization are shown in FIGS. 26, 28, and 30, respectively. These optimized DNA sequences (SEQ ID NOS.: 34, 35, and 36), shown as the lower lines in the figures, were aligned with their original sequences (SEQ ID NOS.: 28, 29, and 30), shown as the upper lines in the figures and the alignments are shown in FIGS. 27, 29, and 31 for rbcL, rbcS, and rpkA, respectively. The genes after the codon usage optimization were de novo synthesized using synthetic oligonucleotides as components. The oligonucleotides were assembled into the appropriate DNA fragments and cloned into the vector pJ201. The cloned DNA fragments were sequence verified in the forward and reverse orientations using a capillary electrophoresis DNA analyzer. The de-novo genes were cloned into the same sets of expression vectors as described above.

While a number of embodiments of the present invention have been shown and described, it will be apparent to those skilled in the art that many changes and modifications may be made without departing from the invention in its broader aspects. The appended claims, therefore, are intended to cover all such changes and modifications as they fall within the true spirit and scope of the invention.

What is claimed is:

1. A method for forming fermentation products utilizing a microorganism having at least one heterologous gene sequence, the method comprising the steps of converting at least one carbohydrate to 3-phosphoglycerate and fixing carbon dioxide, wherein at least one of said steps is catalyzed by at least one exogenous enzyme.

2. The method of claim 1, wherein the microorganism is a type of fungi.

3. The method of claim 2, wherein the fungi is selected from the group consisting of yeasts, filamentous fungi, and combinations thereof.

4. The method of claim 1, wherein the microorganisms are bacteria, archaea, or combinations thereof.

5. The method of claim 4, wherein the bacteria are selected from the group consisting of Zymomonas mobilis, Escherichia coli, and combinations thereof.

6. The method of claim 1, wherein at least one of the exogenous, photosynthetic enzymes is selected from the group consisting of phosphoribulokinase, and ribulose bisphosphate carboxylase.

7. The method as recited in claim 1, wherein the step of converting said at least one carbohydrate to 3-phosphoglycerate further comprises the step of converting ribulose-5-phosphate to ribulose-1,5-bisphosphate.

8. The method as recited in claim 7, wherein the step of converting said at least one carbohydrate to 3-phosphoglycerate further comprises the steps of converting ribulose-1,5-bisphosphate and carbon dioxide to 3-phosphoglycerate.

9. The method as recited in claim 1, wherein the at least one carbohydrate comprises at least one type of five-carbon sugar.

10. The method as recited in claim 9, wherein the five-carbon sugar is xylose.

11. A microorganism for forming fermentation products through fermentation of at least one sugar, the microorganism comprising at least one heterologous gene sequence encoding at least one enzyme selected from the group consisting of phosphopentose epimerase, phosphoribulokinase, and ribulose bisphosphate carboxylase.

12. The microorganism of claim 11, wherein the microorganism is non-photosynthetic.

13. The microorganism of claim 11, wherein the microorganism fixes carbon dioxide.

14. The microorganism of claim 11, wherein the microorganism is a type of fungi.

15. The microorganism of claim 14, wherein the fungi are selected from the group consisting of yeasts, filamentous fungi, and combinations thereof.

16. The microorganism of claim 11, wherein the microorganism is selected from the group consisting of bacteria, archaea, and combinations thereof.

17. The microorganism of claim 16, wherein the bacteria are selected from the group consisting of Zymomonas mobilis, Escherichia coli, and combinations thereof.

18. The microorganism of claim 11, wherein the fermentation products comprise ethanol.

19. The microorganism of claim 11, wherein the sugars comprise five carbon sugars.

20. The microorganism of claim 19, wherein the five carbon sugars comprise xylose.

21. A recombinant yeast comprising one or more heterologous polynucleotide sequences encoding at least one enzyme selected from the group consisting of: phosphoribulokinase, ribulose bisphosphate carboxylase, and combinations thereof.

22. The recombinant yeast strain of claim 21, wherein the heterologous polynucleotide sequences further comprise at least one optimization codon that increases gene expression.

23. The recombinant yeast strain of claim 22, wherein the polynucleotide sequences for ribulose bisphosphate carboxylase large subunit, ribulose bisphosphate carboxylase small subunit, and phosphoribulokinase comprise the sequences set forth in SEQ ID NOs: 34, 35, and 36, respectively.

24. A recombinant microorganism comprising at least one of the DNA sequences set forth in SEQ ID NOs.: 34, 35, and 36.
